(19) United States
(12) Patent Application Publication
(10) Pub. No.: US 2026/0182940 A1
(43) Pub. Date: Jul. 2, 2026

(54) small things in large an long hole collimator for high-sensitivity and high resolution radiation imaging system

(71) Applicant: christian jeanguillaume, Bouchemaine (FR)

(72) Inventor: christian jeanguillaume, Bouchemaine (FR)

(21) Appl. No.: 19/004,288

(22) Filed: Dec. 28, 2024

(51) Int. Cl. A61B 6/42; A61B 6/06; G01T 1/24

(52) U.S. Cl. CPC A61B 6/4208; A61B 6/06; G01T 1/24

(57) ABSTRACT

A high sensitivity, high-spatial-resolution radiation imaging system aimed at performing section images of emitted radiation. This system includes a collimator having at least one large opening to obtain a high-sensitivity. The high spatial-resolution is obtained by the disposition at the entrance of the holes of the collimator of one or several small thing (chip or rods) made in radiation-absorbing material whose base polygon projection approaches with either the shape of the detector pixel or the shape of the reconstructed voxels. The camera head is displaced in motion, composed of scanning exploration and/or rotation around the sources of radiation. Information collected by the detector, combined with information relating to the position of the head of the camera relative to the emitting object are processed to recover the tomographic images. The presence of the aforementioned small things reduces the condition number of the transfer matrix of the system.

### BRIEF SUMMARY OF THE INVENTION

It is the object of the present patent application to provide some improvements to the high-sensitivity gamma camera proposed in the U.S. Pat. No. 5,448,073C. Jeanguillaume September 1995 and U.S. Pat. No. 6,342,699 B1 Jeanguillaume January 2002. The principal improvement is to associate small things of high Z material positioned in the big-hole collimator which allows a more precise knowledge of the data to be acquired and eases the reconstruction algorithm. Better images and a more accurate reconstruction can then be achieved.

### CROSS REFERENCE TO RELATED APPLICATIONS

This application claims the benefit of provisional patent application Set U.S. 63/615,855 Filed Jan. 9, 2024 by the present inventor (jeanguillaume) Title:

Small token, large and long hole collimator for high resolution high sensitivity radiation imaging system.

which is incorporated by reference in its entirety.

### DESCRIPTION OF THE PRIOR ART

Two types of collimators are currently used in the prior art to provide images with gamma rays emitted by the patient after the injection of a single photon emitter. The information provided by the detector (50) combined with the position of the camera head (55) are recorded and processed by a computer. The transfer matrix of usual tomography acquisition-collimation systems presents a huge condition number (meaning a poor result). The slices or tomographic sections are represented (70).

FIG. 3 represents the 2 factors which limit the sensitivity of a thin-hole collimator. The sensitivity is the ratio of the number of detected photons (which reach the detector) to the number of photons emitted by the object. This sensitivity depends on the presence of 2 factors: a solid angle and a surface. The upper left corner of FIG. 3, shows the acceptance angle of one hole of the collimator (30). This solid angle is divided by the 4PI steradians because the radiation emitted by the patient is emitted throughout space. The lower left of FIG. 3 shows the active surface of the collimator versus its real surface (233). Sensitivity is calculated by the product of these two ratios: (230/232) and (231/233).

The two types of collimator used nowadays are:
#1 The very old Pinhole which consist of a very thin hole drilled in a high Z material plate. The hole has a very small diameter (small surface), but the acceptance angle can be quite large.
#2 the so called ‘thin-hole collimator’ which is more often used is made of a large numbers of thin tubes assembled in a plate. This system can cover a wide surface but the acceptance angle is very narrow.

FIG. 2 represents the state of the art with a thin-hole collimator. The radiation emitter (10) emits radiations (20), the said collimator (30) stops the radiations which is not traveling in a path perpendicular to the detector. Typically, for each radiation touching the detector (40), 10,000 radiations are lost. The thickness of the chosen septa separating the holes is very thin to avoid a huge loss of acceptance surface. This thinness reduces further the efficacy of the collimation.

Moreover, the length of the holes in a parallel-holes collimator is generally chosen small (5 to 10 mm) to limit the loss of sensitivity. Letting P be the depth of these holes (FIG. 4-435), the smaller the P the higher the impairment of the spatial resolution with the source to collimator distance. In FIG. 4 the two sources 11 and 12 give gaussian response 451 and 452, the deeper source 12 giving a larger and blurred response (452).

Hence the actual radiation cameras have an important drawbacks:
-/pinhole: very small surface entrance,
-/parallel-holes collimator: very small acceptance angle and reduced surface entrance with faulty collimation.

These collimators suffer from a huge waste of gamma photons. To give an approximate idea between 10,000 and 100,000 photons are lost for one detected photon.

Unfortunately, all the information received by the camera is carried out by these photons. Fewer photons means less information to reconstruct the tomographic images. FIG. 5 shows a very simple image simulated with the detection of a chosen number of photons collected. Despite the simpleness of this example 10,000 photons are needed to recognize the initial object (left part of FIG. 5). These conventional collimators present the user with a severe tradeoff. They can either choose to enlarge the acceptance (surface or angle), which results in more blurred images, or, further reduce the acceptance which leads to more noisy images.

To fully measure the defects of the prior art we need to introduce the ‘condition number’:
-/1. The condition number:

The condition number measures the difficulty of resolving a matrix equation. Deconvolution and tomographic reconstruction can be represented by matrix equation. A large condition number means a difficult problem fraught with a big amplification of the noise during the reconstruction stage. (FIG. 6,10)

This important fact can be illustrated by FIG. 6 in a very simplified system. Let us imagine a 2×2 matrix. The matrix records the slope of the lines. The data give the position of these lines. Solving such a system is like finding the intersection of 2 straight lines. Obviously if the 2 lines are well defined the intersection can be calculated with a high precision. (2 upper parts of the figure). However, when the measure is fraught with uncertainty the result differs significantly in the 2 cases (left and right). In the lower-left part of the figure, the 2 lines cross over each other with an angle of 90°, the intersection point being situated in a limited region of the graph. But, when the angle defined by the 2 lines is small, the uncertainty can reach an unmanageable value. This is illustrated in the lower-right part of FIG. 6. The range of the possible X value is paradoxically almost infinite.

The condition number (CN) is just the ratio measuring the amplification of the uncertainty of the system. In the US

$$CN = Δ re / Δ ac$$

Where Δac is the uncertainty at the acquisition level

And Δre is the uncertainty at the reconstruction level

Therefore, good reconstructed images, means a small Are:

$$Δ re = CN * Δ ac$$

hence to get better images, we need to reduce the condition number CN and possibly the uncertainty at the acquisition (Δac). That is exactly what we propose here. Δac will be reduced by the use of big holes and the CN will be reduced by the use of small things (tokens or rods).

It should be noted that the classical tomographic acquisition with an orbital motion around the patient leads to a transfer matrix with a huge condition number. We already published research (Advances in Molecular Imaging, 2017, 7, 13-47) showing that in a reduced 2D system to reconstruct a single 64×64 image with 128 step angles of acquisition the CN is: 51,255, a 5-figure number!

For a real 3D system, the CN increases with the matrix size. The minimal value of the CN is 1. This corresponds to zero amplification of the noise.

To resume the prior art: we get an unacceptable sensitivity ( 1/10,000) giving a high level of uncertainty. This uncertainty is amplified by a huge condition number. The cherry on the cake: the spatial resolution decreases quickly with the source-to-collimator distance.

One way to greatly reduce the acquisition noise is to increase the number of recorded radiations by enlarging the diameter of the collimator holes. But this used to lead (in the prior art) to a loss of spatial resolution.

In addition to these 2 collimators type a lot of works have been devoted to coded aperture. For example C. Lanza 1999 U.S. Pat. No. 5,930,314 but all this attempts used a mask with a plurality of pinholes. Hence they are fraught with a poor sensitivity. Wilson et al (IEEE TRANSACTIONS ON MEDICAL IMAGING, VOL. 19, NO. 5, May 2000 speak of synthetic-Collimator data but it is just another name for multi pinhole.

### Operation:

The flowchart of FIG. 27 shows 2 columns of steps. The first on the left displays the choices of the parameters needed to prepare an acquisition and a reconstruction of the images. These two operations are schematized in the right column. The following figures (up to 34) detail the calculation of the transfer matrix of the system. The method resembles the one used to calculate the convolution matrix on FIGS. 10 and 11.

The dimensions of the emitting object will determine the extent of the linear scan necessary to study the object in its entirety. This point is illustrated in FIG. 28. To get an accurate reconstruction of the image it is better to use a linear scan whose extreme steps go as far as a complete shadowing of the detector. Of course an acquisition which provides no data is useless, so we recommend stopping just one step before. If one big hole is necessary, the more the merrier, as illustrated by FIG. 19. If we choose to use more than one big hole, these holes may be similar (with the same number, arrangement and shape of small things) in order to reduce the scan. They can also present a different arrangement of small things, in order to reduce the condition number. The embodiment of FIG. 28 shows a collimator with 2 big holes with the same arrangement (1 small thing). The collimator is represented in two extreme positions (left and right), to ensure a good condition number. The two extreme rays (500,510) show the complete extinction of the signal. It is also possible to use a plurality of big holes with different arrangements of small things. In this case a complete scan should be done with each arrangement.

Once all the parameters are chosen, it is possible to calculate the transfer matrix of the system. FIG. 30 shows the principle, which mimics the construction of the convolution matrix depicted in FIG. 10 and FIG. 11. We begin with a relative position of the object and the collimator-detector. We choose a point for this object and we determine the image recorded by the detector. FIG. 9 and FIG. 31 show how to draw the shadow of a small thing on the detector. Giving a source, in the case of a square base object the 8 corners of the object will project on 8 points at the corners of 2 squares. The face of the object (110) nearer to the source will project on a bigger square (116) than the face nearer to the detector which provide a smaller square (117). To draw the shadow you keep the 6 exterior points in order to have a convex figure with at most 6 sides (115). Depending on the relative position between the source and the small thing, the number of sides can be reduced to 4 or 5. For example if the source is situated on the center of symmetry of the small thing the small square (117) will be totally hidden by the bigger square (116).

To produce the transfer matrix we need also to take into account the shadow of the side wall of the big hole. This is illustrated by FIG. 32 which shows a cut detector (5×5 pixels) The front wall and the first row of the pixels have been removed. The source projects a shadow of the farther and the left sidewall. FIG. 33 shows for the same arrangement the drawing of the shadow of the small thing.

FIG. 34 shows the computer result of the image recorded on a detector (5×5) for a source placed at a small distance from the upper left corner of the detector.

FIG. 34.A shows the hypothetical enlightenment that the detector would receive in the absence of any collimator. This can be calculated by dividing the brightness of the source by the source-to-detector distance to the power 2, and multiplying by the cosinus of the angle between the ray and the perpendicular to the detector surface. This cosinus term is easy to understand when you look at the temperature of the earth which is warmer at the equator (ray angle:) 90° than at the poles (larger angle of the sun rays)

FIG. 34.B shows the limits of the shadow produced by the walls of the big hole, and the limit of the shadow on the small thing. This FIG. 34.B allows the calculation of the percentage of surface enlightenment of each detector pixel.

FIG. 34.C shows the results of this percentage applied to FIG. 34.A. Note that FIG. 34.A and FIG. 34.C are represented in greyscale from the maximum of each figure to the minimum of each one. The minimum enlightenment is represented in black and the maximum in white. Observe that the variations of enlightenment in FIG. 34.A are weak compared to that of FIG. 34.C. The pixels on the right lower border of the detector exhibit no visible grey in the FIG. 34.C.

Once the enlightenment image is done, it represents a line of the transfer matrix. This line is formed by all the rows of the calculated image. FIG. 35 represents how the image is cut into stripes to give the line of the matrix. FIG. 36 shows some calculated images in a linear scan.

How to inverse this matrix and calculates the tomographic images of an acquisition? A number of procedures have been proposed in the literature. For the calculation of FIGS. 14,17,18,20 we uses a Penrose pseudo inverse. FIG. 45 give the formula of the pseudo inverse. Better results may be obtained with MLEM, and other regularized methods. But for the comparison the priority was to use the same method.

To fully described the invention, if A is the transfer matrix At its transpose and −1 the inverse of a square matrix The pseudo inverse A* is:

### Advantages:

We already demonstrated in 2D that the tomography deconvolution with large holes exhibits a better condition number than the conventional thin hole collimator and the radon transform. Here we show that the 3D acquisition with large holes and small things presents a greater sensitivity and a better condition number than without small things. And we show that with an increasing number of small things we can obtain even lower condition number. Finally with the use of more big holes we continue to reduce the condition number while maintaining a high sensitivity. We said that the condition number eases the reconstruction and reduce the amplification of the noise but it is also known to increase the reliability of the reconstructed images. Further more a low condition number provide a more precise spatial resolution.

This will provide better sensitivity and better spatial resolution with sharper and better defined images.

All the physical advantages mentioned earlier will benefit the end-users. In the medical field that means physician and patients. The shift of the compromise regarding spatial resolution vs sensitivity will lead to better diagnostics and better health. The increased sensitivity can be used to:
1) reduce the examination time
2) reduce the blur due to involuntary patient movement during the examination,
3) increase the throughput of the team
4) reduce the injected radio activity (hence reducing the radiation burden of the patient.
5) reduce the degradation of spatial resolution linked to the detector-source distance.
6) increase the quality of the reconstructed images, hence in the medical field: improve diagnostic quality and power.

### Disadvantages:

The necessity of performing a complete scan (linear or orbital) obliges to study static object or slow evolving object. The evolution of the object must be slower compare to the duration of the examination. Another possibility stands for a periodic evolving specimen where gated acquisition can be performed.

### BRIEF DESCRIPTION OF THE VARIOUS VIEWS OF THE DRAWINGS

FIG. 1 is a schematic view, one embodiment of the high sensitivity radiation imaging system. The source of radiation (or object) 10 emits radiations 20 which enter a collimator 130, the selected radiations 140 are detected by a position sensitive detector 150. A computer 160 inverses the transfer matrix which links the data recorded by the detector 150 and the recording movement of the head of the camera 155 to produce the reconstructed slices 170.

FIG. 2 is a schematic view of the prior art of the gamma camera with a parallel thin-hole collimator. The source of radiation (object) 10 emits radiations 20 which enter a parallel thin-holes collimator 30. The selected radiations 40 are detected by a position-sensitive detector 50. A computer 60 inverses the transfer matrix which links the data recorded by the detector 50 and the recorded movement of the head of the camera 55 to produce the reconstructed slices 70.

FIG. 3 is a description of the factor used to calculate the sensitivity of a collimator: the acceptance angle 230 in steradians is divided by 4n steradians 232. The useful surface of the detector 231 is divided by the total surface of the detector 233.

FIG. 4 is a schematic view of the loss of spatial resolution with the source-to-collimator distance in the prior art. This loss increases with the use of small length holes 435. Two sources 11 and 12 give 2 responses: 451 and 452 on the detector.

FIG. 5 is an illustration of the benefit provided by recording a higher numbers of photons to produce better images.

FIG. 6 is an illustration of the condition number in a very simple 2×2 matrix. Left small condition number, right awkwardly high condition number. Top row: in exact calculation the crossing point is well defined. Bottom row in the presence of noise the solution is badly delimited by the grey area.

FIG. 7 is an exploded view of a big opening 100 of the collimator in one embodiment, showing 3 things 2 chips 110 and one rod 111 which may be of different shapes. These chips or rods are made from high absorbant material. They are maintained near the entrance plane of the collimator hole through the use of low Z material (transparent to the radiation considered, not represented). These said things are positioned in front of the pixel 115 of the detector 150. Positioned at a distance from the collimator in this exploded view, the source of the radiation is not shown here but considered at an infinite distance.

FIG. 8 shows a sectioned collimator and detector in one embodiment, with the source of the radiation 10. A small thing: rod 111 and a token 110 are also represented to visualize their position in relation to the source and the detector.

FIG. 9 shows how the said thing 110 produces a shadow 112 on the surface of the detector 120. For a small thing with the shape of a square base prism the shadow is formed by two squares of different sizes, joined by two oblique lines. This figure shows that the shadow produced by the small thing and rays coming from a point source 10 can partly fit the limits of the detector pixels (hatching).

FIG. 10. shows how to construct a transfer matrix of 2D convolution. This situation appears when you want to record the emission of a plane emitting source of radiation with a collimator-detector larger than the required spatial resolution. This is possible using surface scanning with a small step (in the order of the targeted spatial resolution). The very simple object has 3×3 pixels, represented by a square in a plain line. The collimator-detector set has a surface equivalent of 2×2 pixels and is represented by a square in a dashed line. 16 relative positions (4×4) of the collimator-detector and of the object are needed in order to acquire and reconstruct the image of the object.

FIG. 11. shows the transfer matrix of the simple example of convolution described in FIG. 10. The 16 rows of the matrix correspond to the 16 scanning steps of the acquisition. The 9 columns correspond to the object studied symbolized above the matrix by 9 pixels gathered 3 by 3 according to the first, second and third lines (from top to bottom). In a matrix line corresponding to a relative position (detector object) we put a 1 in the matrix if the detector can see the pixel in the corresponding column.

FIG. 12. is an illustration of the operation of convolution (80) described in FIG. 11 and of its inverse: the deconvolution (90). On the upper left an image a 3×3 image (the object) is convoluted by a 2×2 square kernel. On the bottom line, the 4×4 result is then deconvoluted by the same kernel to recover the initial image. Note that the convolution operation is symbolized by an X superposed on a circle (80), and we choose to symbolize the deconvolution by a circle with 2 slashes (90).

FIG. 13. is an illustration of the operation of convolution and of its inverse in a larger dimension to show the conservation of image structures. On the upper left an 18×18 image (the object) is convoluted by a 5×5 square kernel. The 22×22 result is then deconvoluted by the same kernel to recover the initial image. This recovery is an exact mathematical operation in the absence of noise.

FIG. 14. shows the same operation as FIG. 13 but we have added a slight noise to the convoluted image before the deconvolution. The upper line is similar to the lower line of FIG. 23, to show the resemblance of the 2 images on the left hand side. However due to the noise amplification of the deconvolution the 2 images in the right hand column look very different. The greater the condition number the greater the degradation.

FIG. 15. is an illustration of single big-hole motion in a 2D plane mimicking a 2D convolution acquisition.

FIG. 16 is an illustration of the same scanning motion as FIG. 15 with a single big-hole but associated with said small thing in a 2D convolution acquisition.

FIG. 17 Shows 2 curves: in black discs the condition number of the 2D convolution matrix for various square holes width (5,10,15,20,32,48,64). In abscissa: the surface of the holes is given in pixels: (25,100,225,400,1024,2304,4096). The grey triangles represent the condition number of the same matrix but with 1 pixel obscured. The points of the second curve are slightly shifted toward the left to show the slight reduction of the useful detector surface: the new surfaces are: (24,99,224,399,1023,2303,4095).

FIG. 18 The black discs shows the evolution of the condition number of the 2D convolution matrix for a square hole (5×5) and a 10×10 object for different numbers of blind pixels in the detector. This mimics an equivalent number of said small things. The first disk is for a square without any blind pixels.

FIG. 19 Shows the comparison of the condition number of a convolution matrix for 1 versus 2 associated big holes. The left column shows the condition number for a single big hole. The right column shows the results obtained with the use of 2 big holes with the same number of small things. The object is a 10×10 square image. The number of small things are 1,2,3 for the 1st line, 2nd and 3d line. Note that the diminution of the condition number here is not associated with a diminution of the useful detector surface.

FIG. 20 shows the influence of the condition number in the image processing of noisy data. Both columns show the result of noise generation followed by a deconvolution. The left column shows the results obtained in a single big hole with a 5×5 square (and no blind pixels). The right column is obtained with the configuration of FIG. 19 last row on the right: 2 5×5 holes with 3 obscured pixels.

FIGS. 21, 22, 23, 24 explain the acquisition sequence in some embodiments studied in the following figures.

FIG. 21 shows a scanning motion in a single horizontal plane.

FIG. 22. shows a scanning motion in a single vertical plane.

FIG. 23. shows a scanning motion in 2 vertical planes and 2 horizontal planes around the object.

FIG. 24. shows a scanning motion in 8 planes around the object.

FIG. 25 shows the sorted range of the condition numbers obtained for different positions of the darkened pixels (5×5 detector) for the 3D tomographic (upper 2×2×4 object) and for a simple 2D deconvolution (lower 4×4 object)

FIG. 26 shows the same condition numbers as in FIG. 25 represented in grey scale in place of the darkened detector pixel, 3D upper, 2D lower.

FIG. 27 represents the general flowchart of how to operate the process in most embodiments. The left part of the figure is needed to initialise the process. When they it is done The acquisition and the reconstruction of the tomograms can be performed without repeating the steps in the left part.

FIG. 28 shows the limits of a linear scan. To produce sufficiently-good set of data to produce precise reconstruction images, the linear scan must comprise the 2 limit acquisitions represented here. The two extreme rays (first 500 and last 510) emitted by the object and traveling from the extreme corners of the big holes are represented.

FIG. 29 shows the flowchart of the preliminary choices needed to calculate the transfer matrix of the system.

FIG. 30 details the different steps needed to calculate the transfer matrix of the system.

FIG. 31 shows a reminder of FIG. 9, for a small thing with the shape of square-based object the shadow is formed by two squares 116 and 117. These two squares are joined by two oblique lines. The projection of the corner of small thing A and B which are inside the figure are discarded to form a polygone of 6 sides delimiting the shadow. This irregular hexagone is represented on the upper right and the lower figure represents this shadow.

FIG. 32 shows the limits of the shadow produced by the side walls of the big hole.

FIG. 33 shows the shadow produced by a single rod placed in front at detector cell number #6. The cells are numbered from left to right and from top to bottom. This arrangement will be kept unchanged for the following FIGS. 34,35.

FIG. 34 illustrates the following steps of the calculation of the transfer matrix. Considering a detector of 5×5 pixels, a source is placed in front of the detector cell 0 (upper left corner of the detector). On the left part (34.A) we show in greyscale a representation of the illumination of the big hole (in the absence of collimator). On the upper right of the FIG. (34.B) we have drawn the limit of the shadow due to the side wall (FIG. 32) and the hexagone delimiting the shadow of the small thing (FIG. 31). The right lower part of the FIG. 33) represents the final illumination in grey scale 120. That is the illumination of (34.A) diminished for each detector cell by the two shadows (side wall+small thing).

FIG. 35 shows how the detector illumination image 120 is sectioned in line and the line is assembled (concatenated) to produce a vector.

FIG. 36 shows some of the images in grey scale 120 calculated in one linear scan. The complete set run from the abscissa-12 to +12 to obtain a complete extinction. But only 6 abscissa are represented. The first column shows the abscissa, the second column the limits shadow, of the side wall and the small thing. And the third column represents the final illumination images in grey scale.

FIG. 37 represents the final formation of the transfer matrix: all the precedent vectors are assembled in a matrix including all the hole sets, all the angles of rotation steps and all the linear scanning steps, for all the different arrangements of small things chosen. The order of the different steps is not important.

FIG. 38 shows another advantage of the small thing. This figure demonstrates that the shadow of the small thing or rod gets smaller when the source of radiation is farther from the collimator. The blur inherent to the prior art has vanished here. The right part of the figure shows that in some embodiments, the small thing may be thinner than the larger of the detector cells in order to fit the boundary of the detector cells.

FIG. 39 should be compared to FIG. 4 (prior art) In FIG. 4 we saw that the response function of the point source is a Gaussian which keeps enlarging with the source to collimator distance while the intensity is decreasing. In the invention described, the response is becoming thinner maintaining a high contrast. The intensity is also decreasing but at a lower rate.

FIG. 40 presents another embodiment where several big holes and detectors (here 3×6) are arranged in slanted lines in a set. This assembly allows for a dense scanning motion while diminishing the number of scan lines.

FIG. 41 shows possible embodiments for the small things. The rectangular parallelepiped shape can, for a better shielding presents a pyramidal entrance or exit face, (upper center) or a convex curved shape (right upper part of the figure). The base of the prismatic could be any shape: polygonal with any number of sides.

FIG. 42 shows another possibilities for the small things: an icosahedral, a sphere, a cylinder or an ellipsoid

FIG. 43 shows a possible embodiment where the resulting relative motion of the camera head (collimator+detector) is helicoidal (improperly called spiral). This can be obtain with a maintained circular motion of the camera head while the object perform a smooth linear scan.

FIG. 44 shows the comparaison of the condition number (CN) obtained in 2 situations. One a 2D arrangement with a 5×5 detector in front of a 4×4 image. Second a 3D arrangement with a 5×5 detector big hole studying a 2×2×4 object.

FIG. 45 formula for the Penrose Inverse

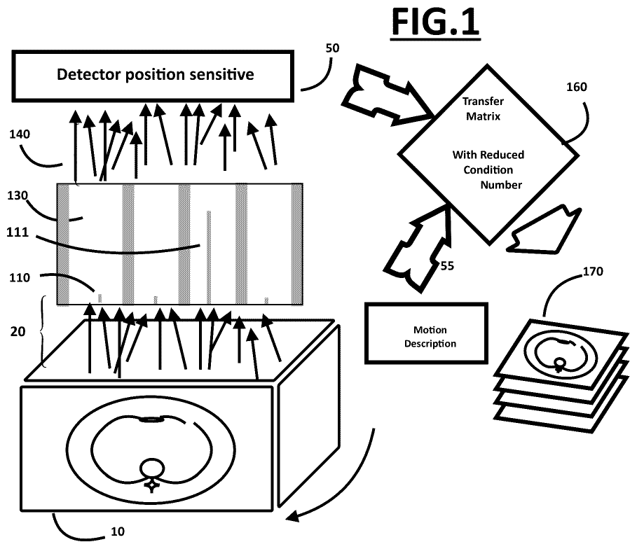

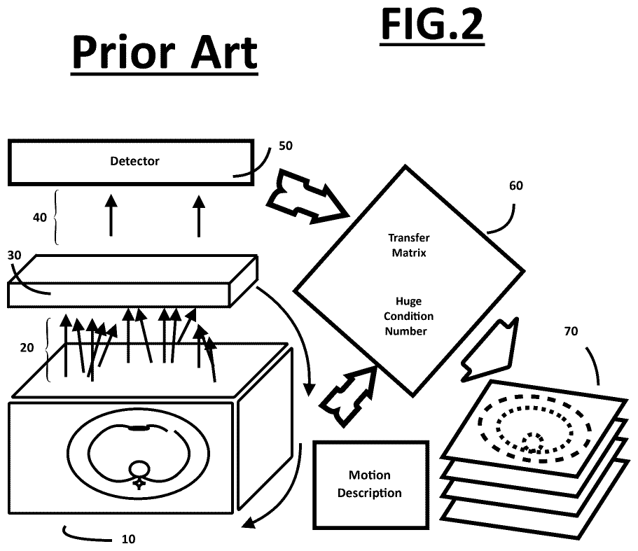

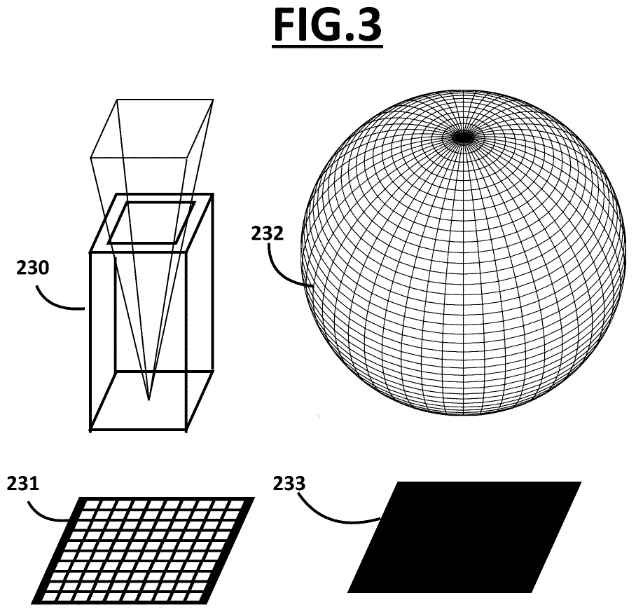

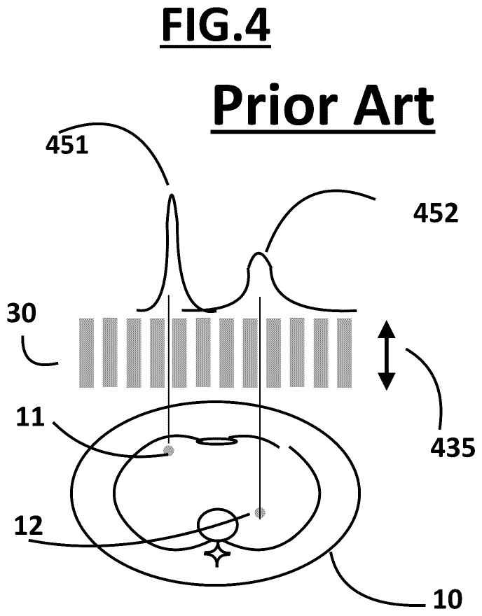

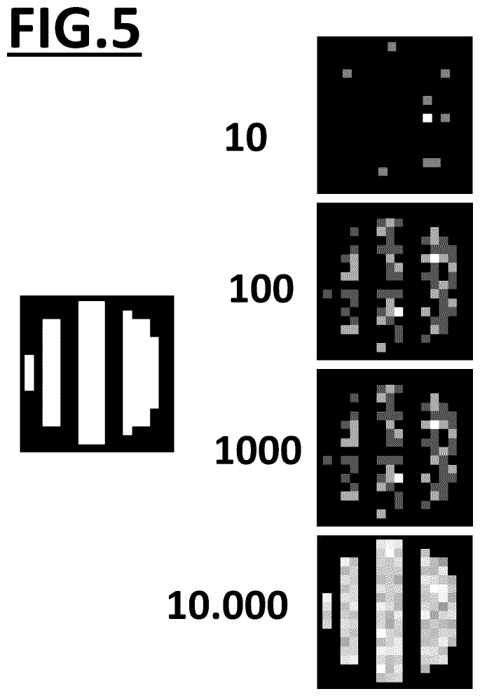

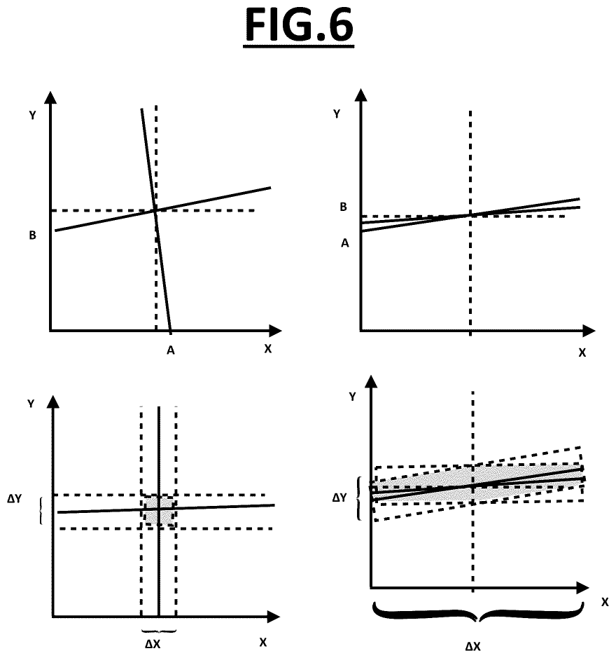

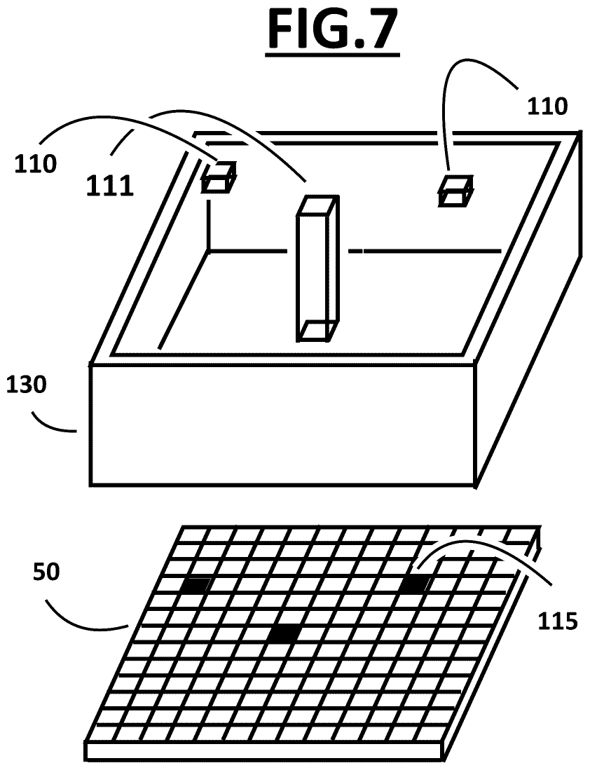

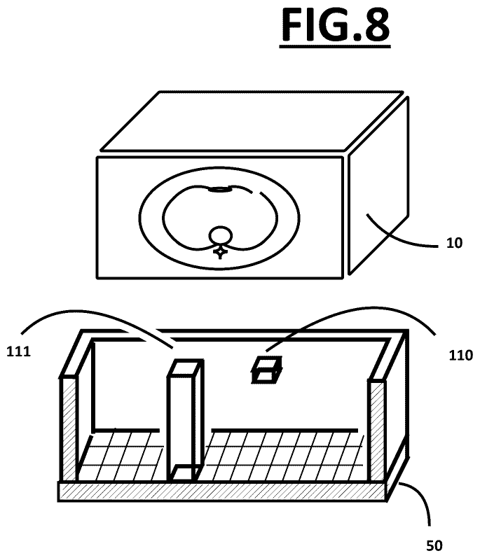

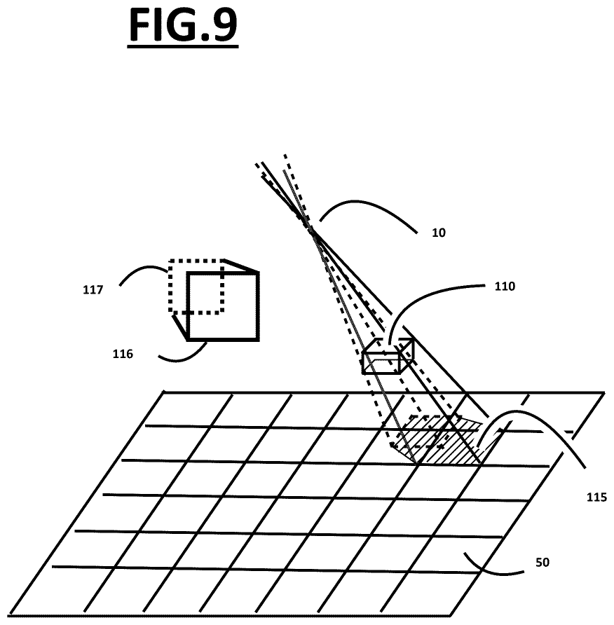

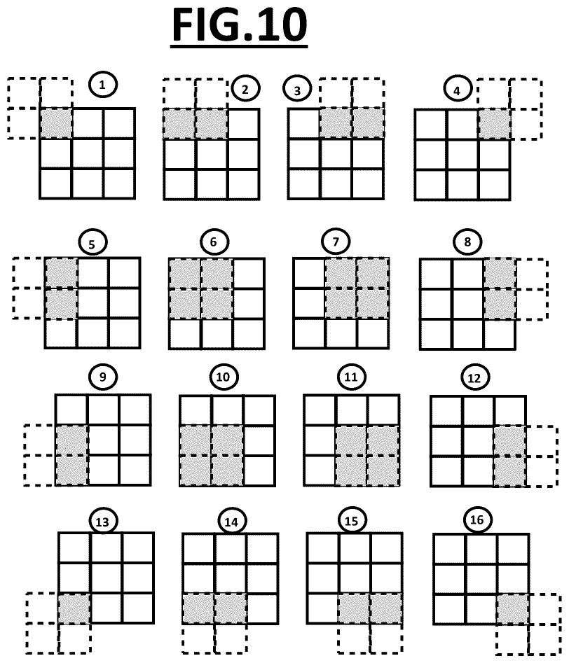

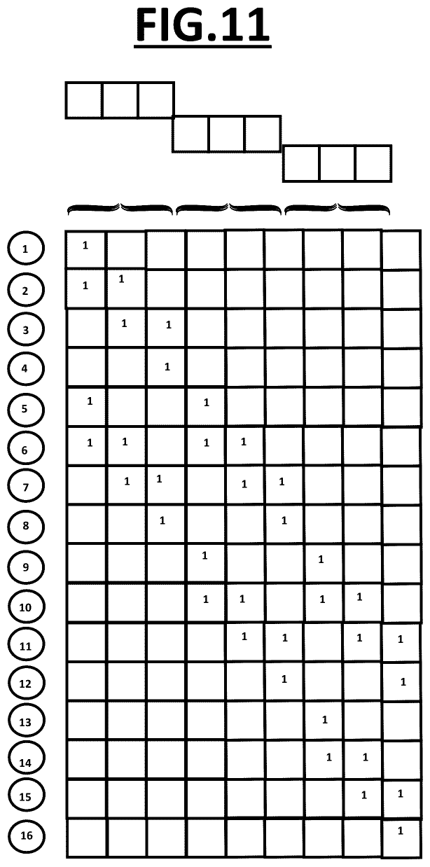

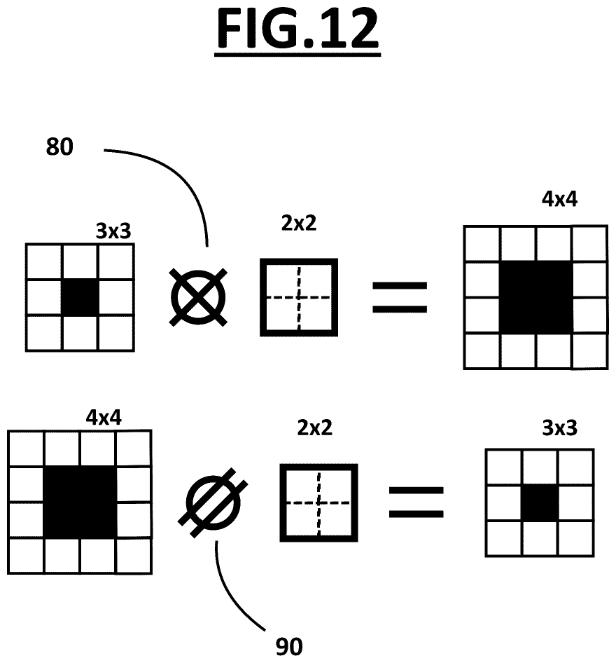

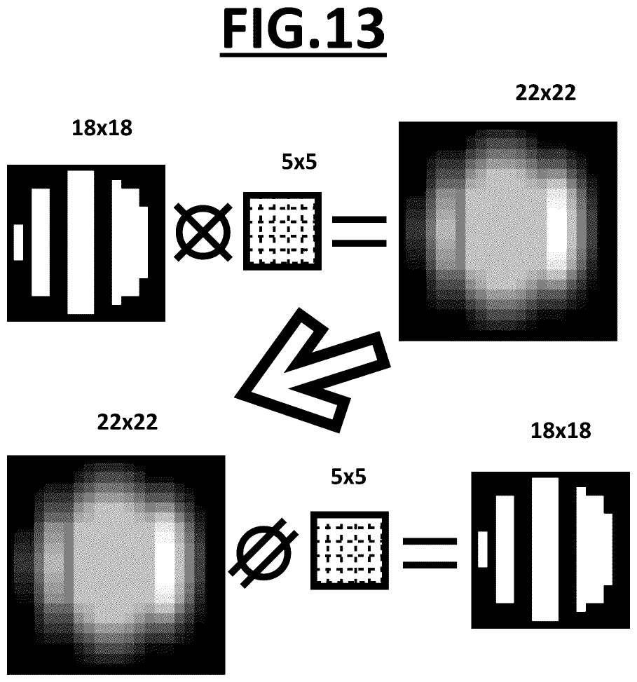

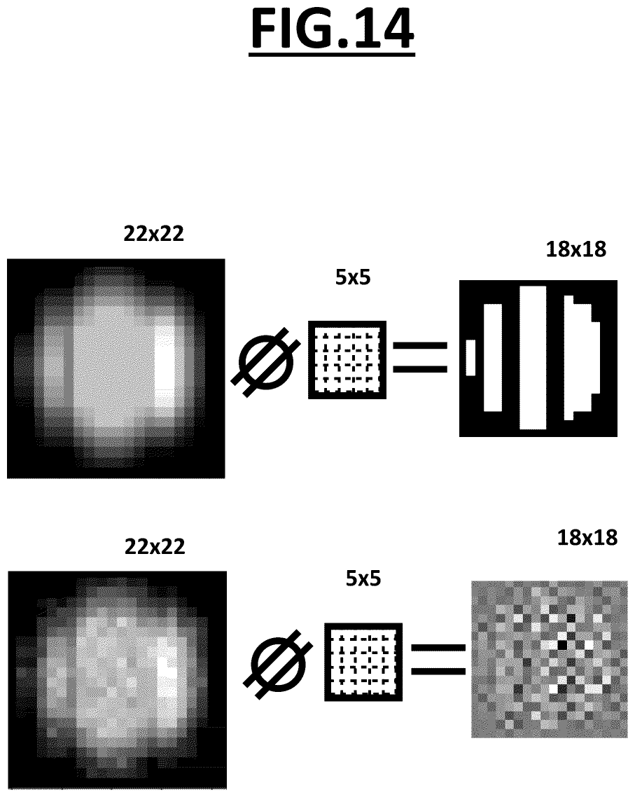

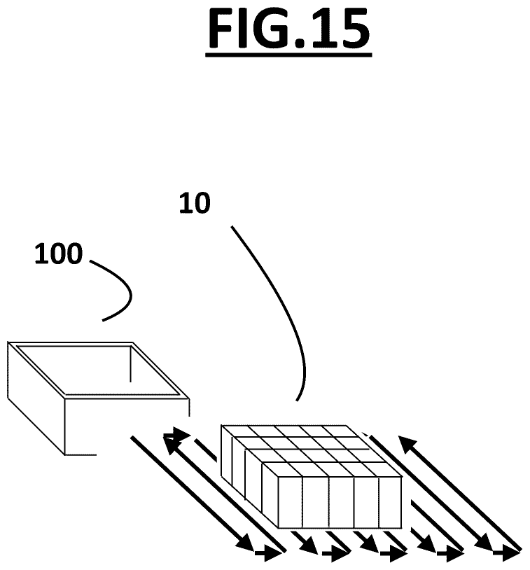

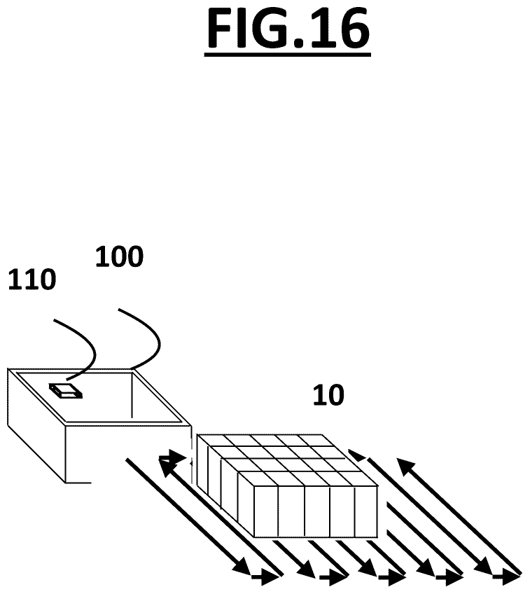

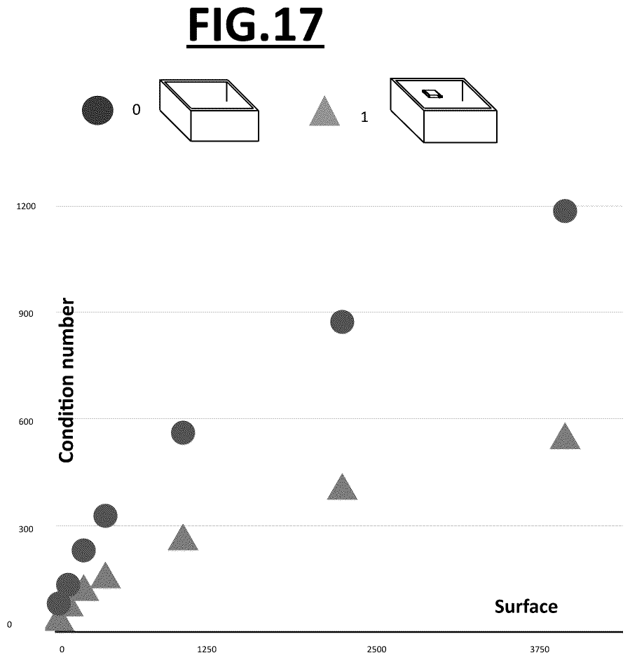

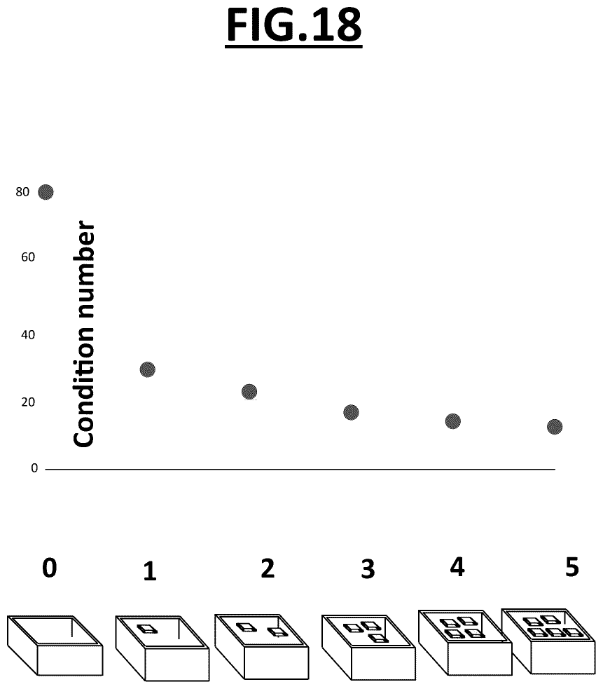

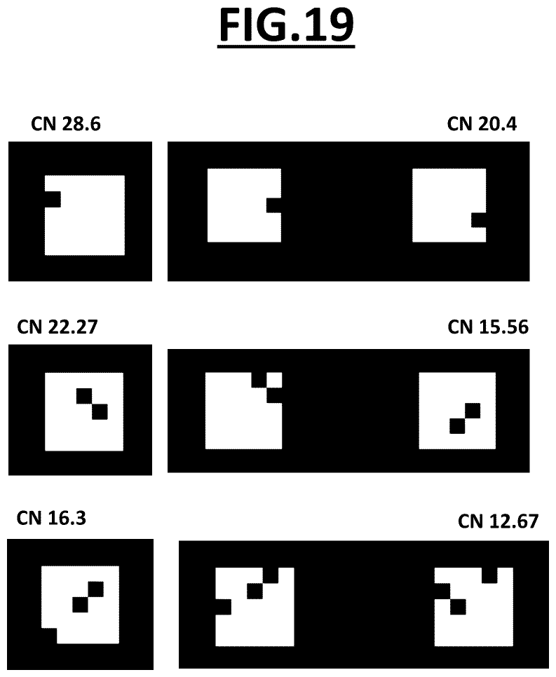

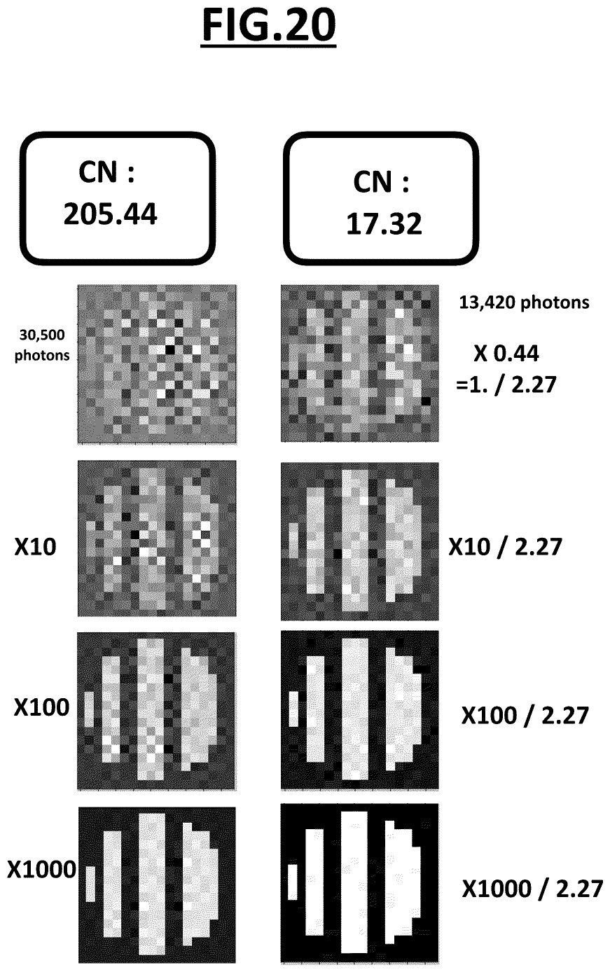

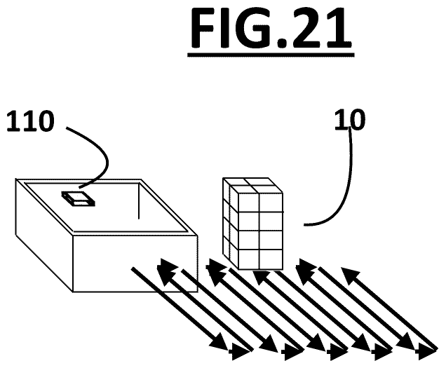

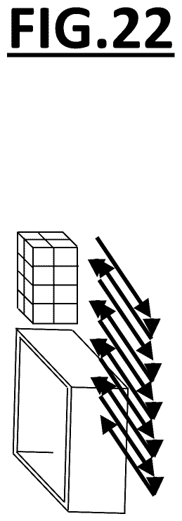

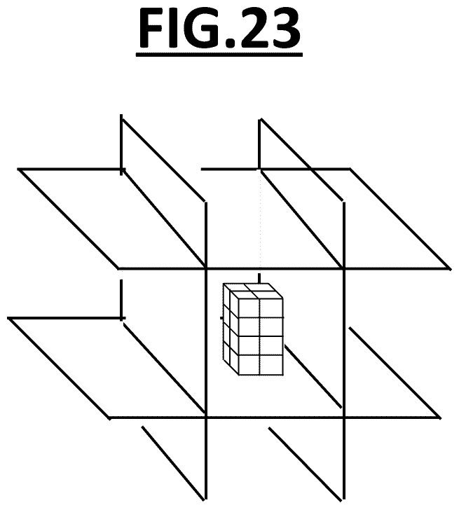

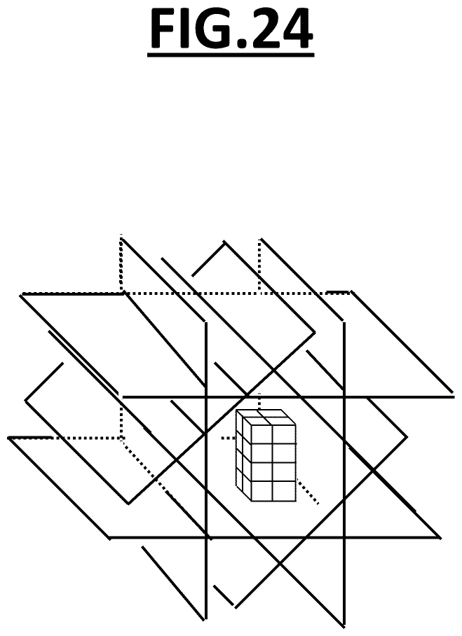

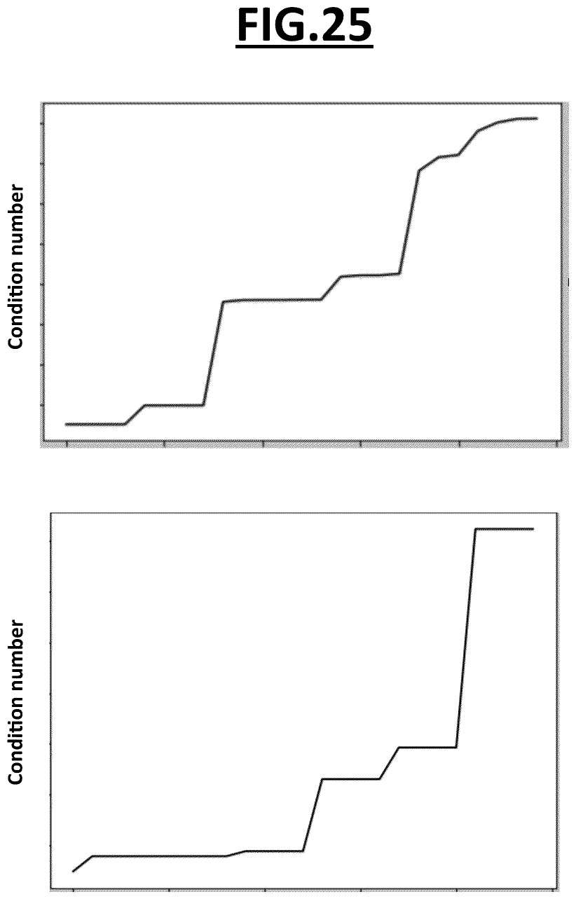

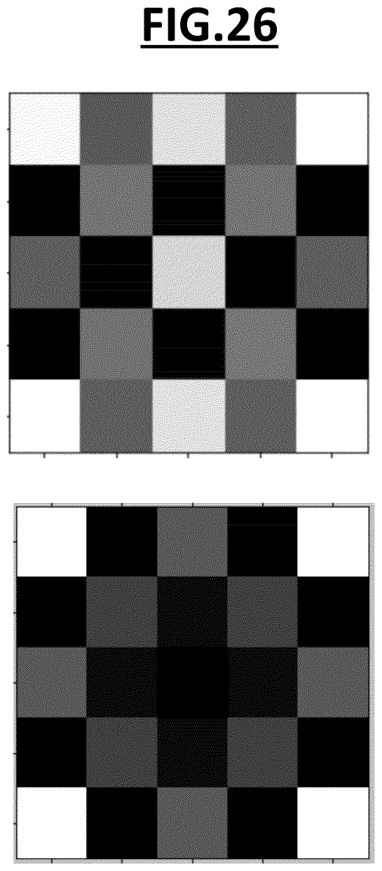

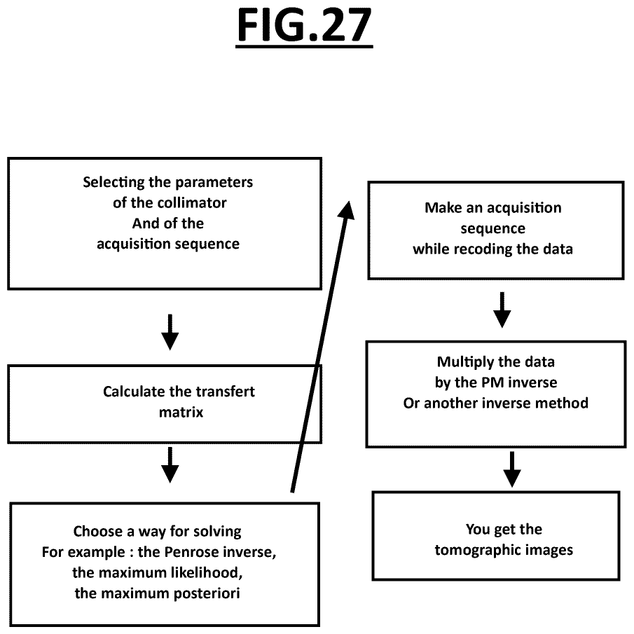

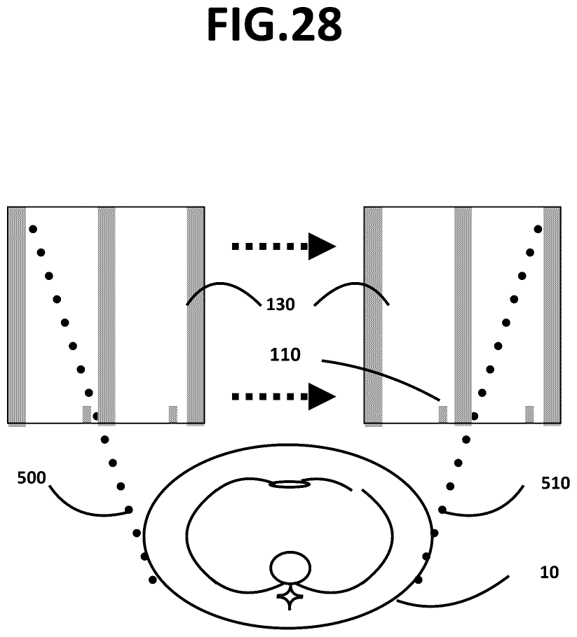

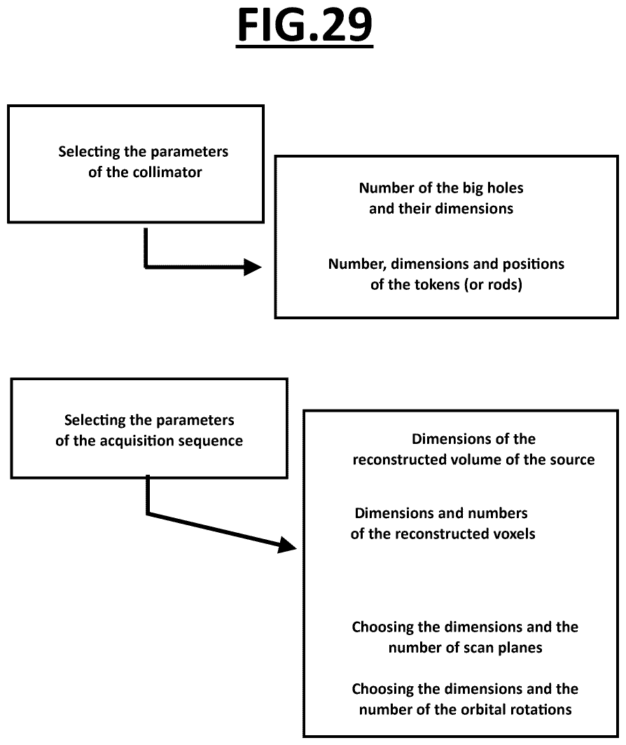

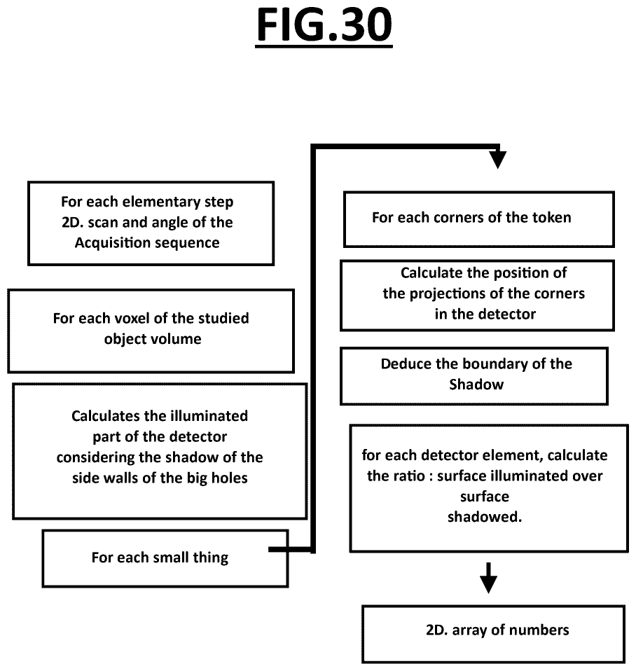

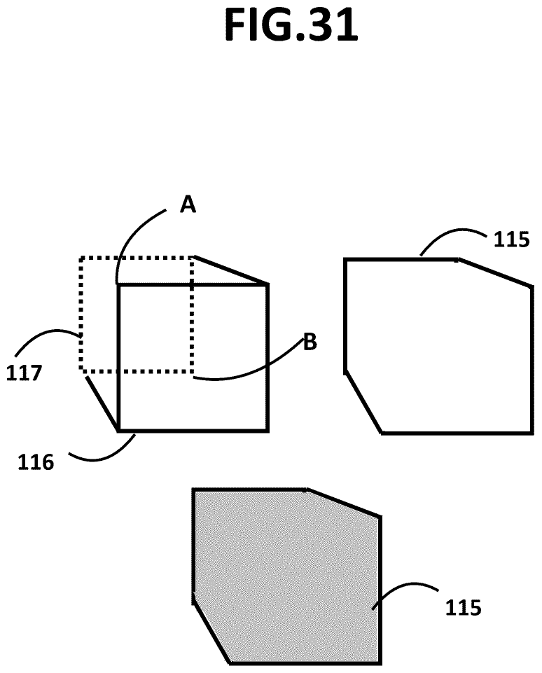

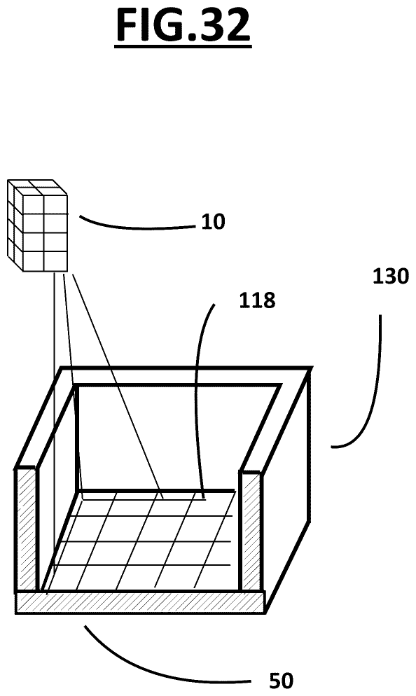

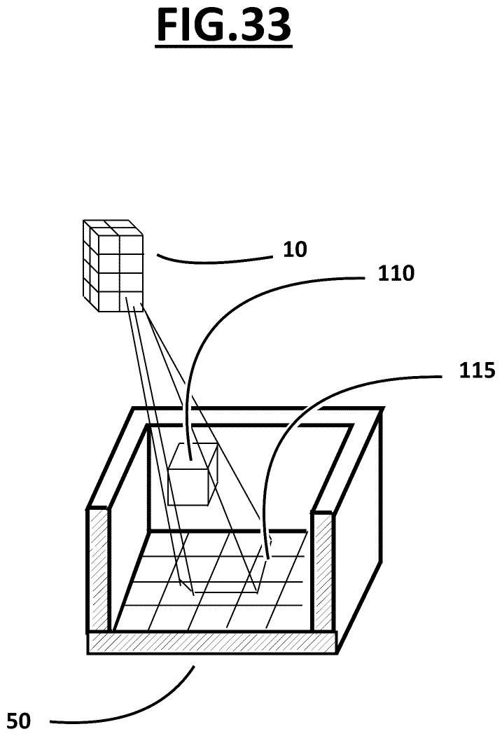

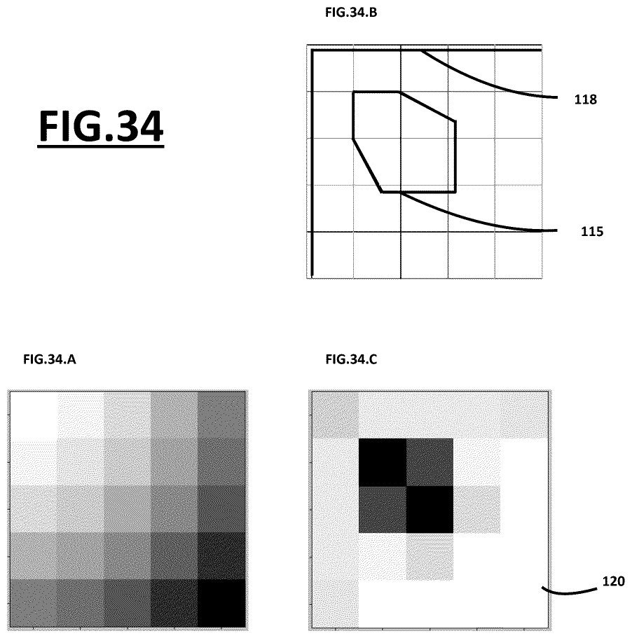

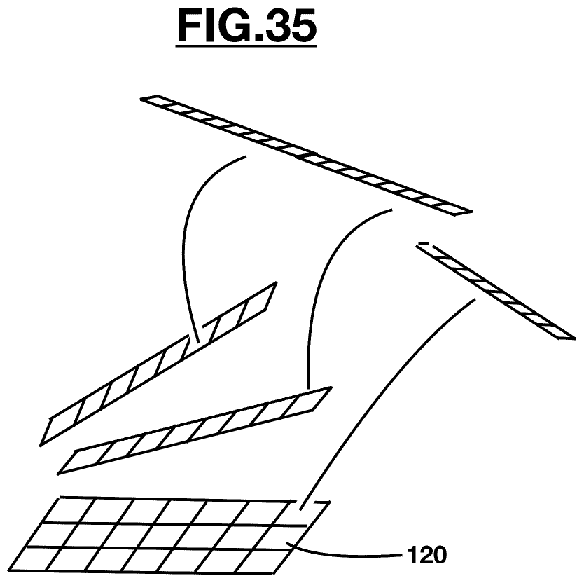

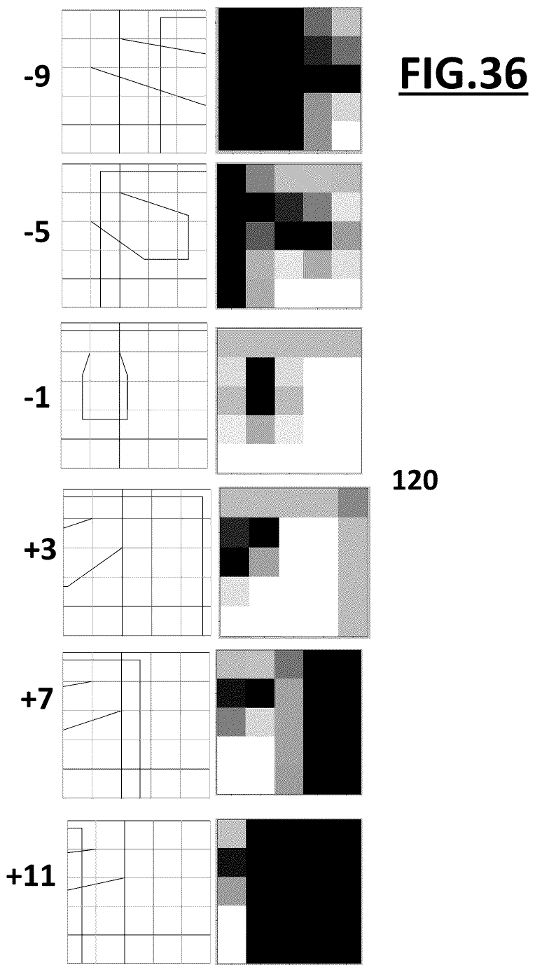

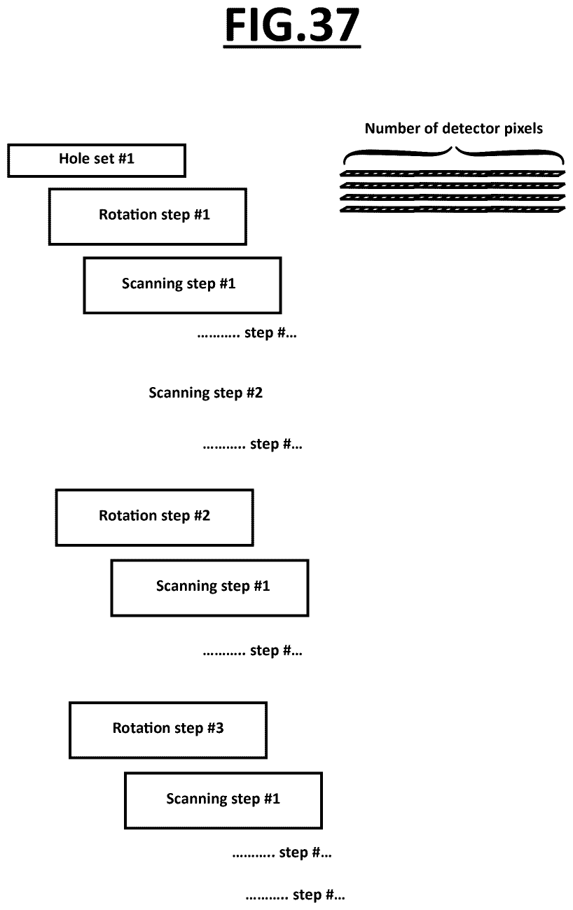

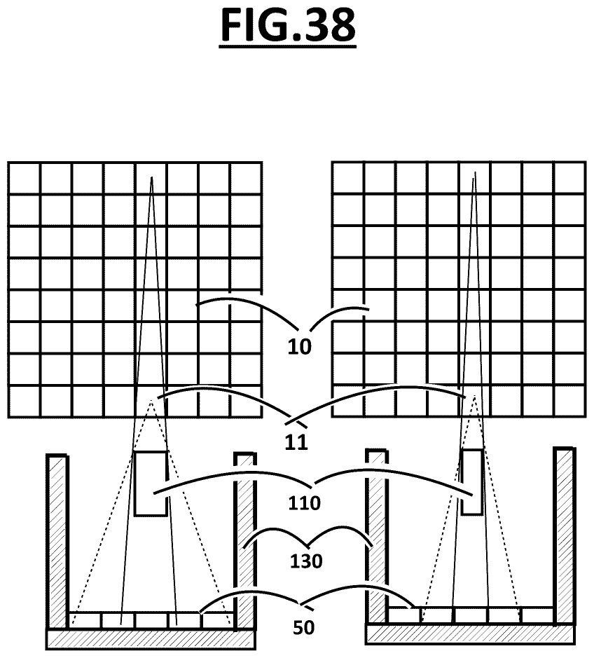

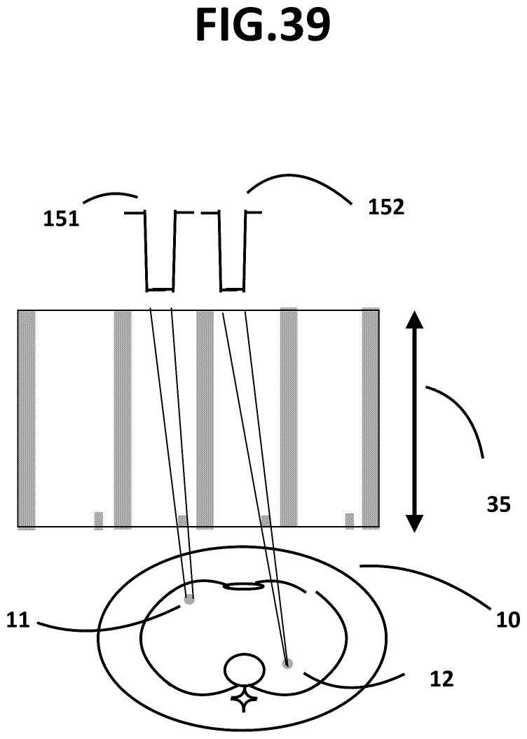

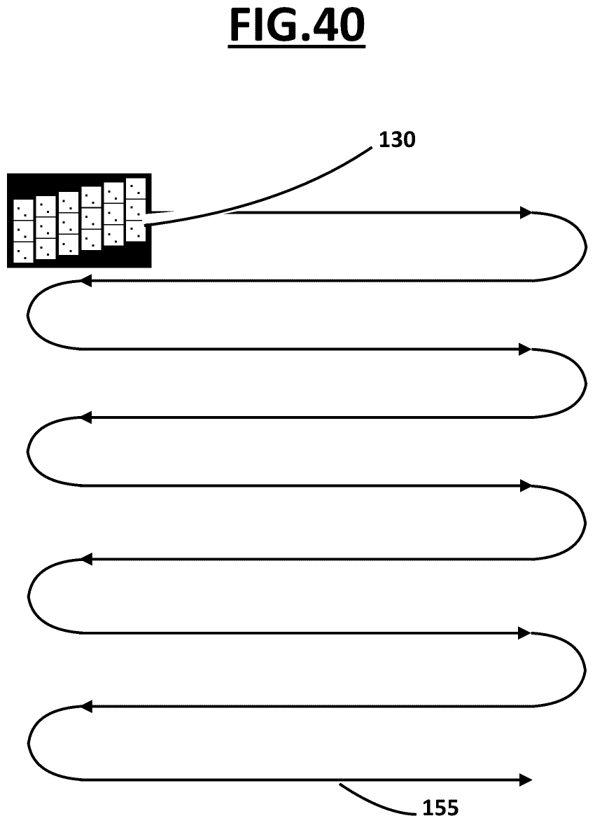

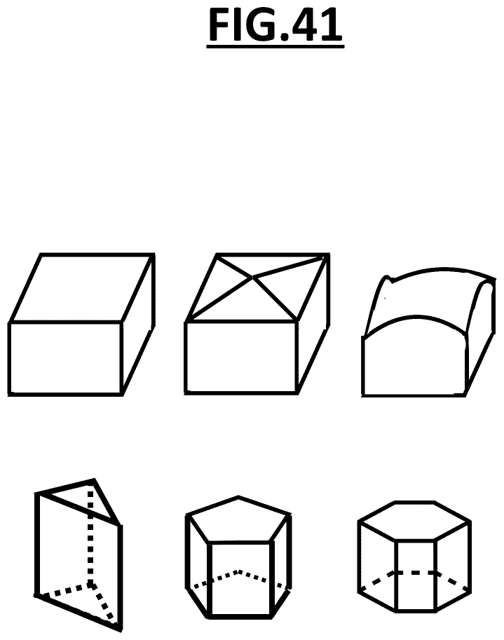

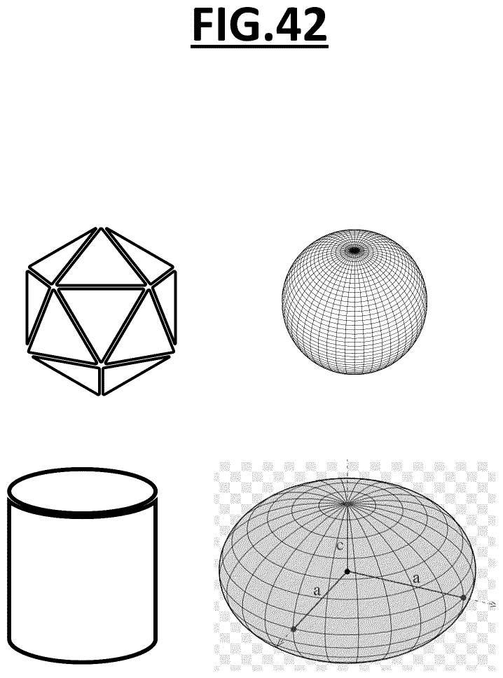

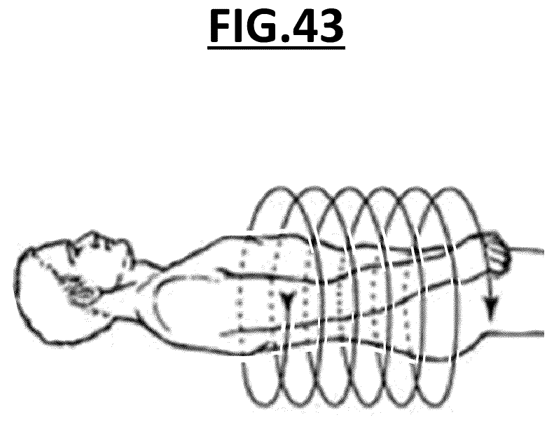

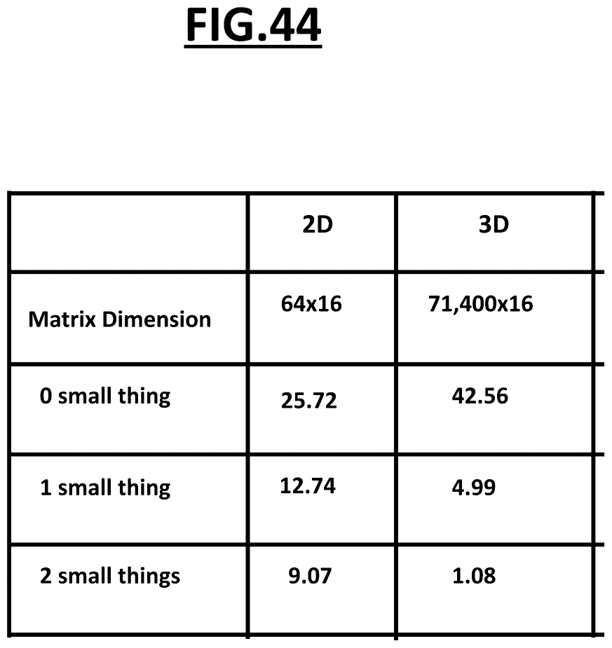

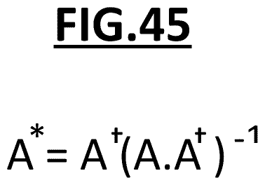

### ETIQUETTES LISTE

10 object emitting radiations
11 point of the said object nearer from the detector
12 point of the said object farther from the detector
20 radiations emitted by said object
30 conventional thin hole collimator
40 radiations selected by the classical thin hole collimator
50 radiation detector position and energy sensitive (can be pixelated)
55 computer managing the motion of the detector relative to the object
60 computer inverting the transfert matrix of the classical tomographic system (With a huge condition number)
70 set of tomographic images reconstructed
80 symbol representing the convolution operation
90 symbol representing the deconvolution operation
100 side wall of the big hole
110 small thing: token or chip made with absorbant material
111 small thing: rod that may takes place in another embodiment
115 limit of the shadow produced on the detector by small thing: 110 or rod 111
116 larger square delimiting the shadow of the small thing face nearer the source
117 smaller square delimiting the shadow of the small thing face nearer the detector
118 limit of the shadow produced on the detector by the side wall of the big hole
120 image recorded on an acquisition step
130 large and long hole collimator
140 large set of radiations selected by the said collimator (130)
155 path of the set collimator-detector during the scan in one embodiment
160 computer inverting the transfert matrix of the said tomographic system (With reduced condition number)
170 set of tomographic images reconstructed by the said invention
230 one hole of a conventional thin hole collimator (30)
231 useful surface of the detector in front of the holes
232 representation of the 4π steradians
233 representation of the total surface of the detector
411 point of the object emitting radiations
412 point of the object, like (411) but farther from the detection system
435 depth of the conventional thin hole collimator (30)
451 response of the point (411)
452 response of the point (412)
500 extreme ray delimiting the beginning of a linear scan
510 extreme ray delimiting the ending of a linear scan

What is claimed is:

1. High-sensitivity high-resolution radiation imaging system aimed at providing sections of three dimensional density of an emitter of an object comprising:
(a) a detector sensitive to the position and the energy of the impinging radiation and having an intrinsic spatial resolution, the said detector converting the position of impinging radiation into electrical signals,
(b) a collimator having one or more big openings with the entrance face directed towards the object to be imaged, and an exit face oriented towards the detector, these openings having narrowest cross-section several times larger than the spatial resolution,
(c) several small things or rods at the entrance face of the said big opening made from a highly absorbent material, aimed at projecting a shadow onto the detector, mimicking the shape of the detector cell or the voxel to be reconstructed,
d) the cross section of the said small things measured in a direction parallel to the detector is smaller than 1.5 time the spatial resolution,
(e) a means of holding the source of radiation to map and to move the said collimator and the said detector in a plurality of movements, said movements are defined relative to the source,
(f) a means of conducting the said movements, of storing the said electrical signals related to the position of the said collimator and the position of the said radiation impact, of processing the information thus stored in order to reconstruct the image of the said emitting object.

2. The radiation imaging system according to claim 1 wherein said movements are conducted until the step before the total extinction of the collected radiation.

3. The radiation imaging system according to claim 1, wherein said different big openings are arranged in different sets and in each set having a special arrangement of small things.

4. The radiation imaging system according to claim 1, wherein said openings lie on mutually parallel straight lines these straight lines make an angle with the direction of the said scanning movement such that the shift of the centers of two successive openings cover the scanning plane in a rectangular or triangular mesh.

5. The radiation imaging system according to claim 2 wherein the said movements are linear scans.

6. The radiation imaging system according to claim 1 wherein the said movements are circular or elliptic all around the said emitting object.

7. The radiation imaging system according to claim 1 wherein the said movements are linear scans performed in 4 or 8 planes around the said emitting object.

8. A radiation imaging system according to claim 1 where the scanning mesh fits a plurality of squares.

9. A radiation imaging system according to claim 1 where the scanning mesh fits a plurality of equilateral triangles.

10. A radiation imaging system according to claim 1 used to record gated data.

11. A radiation imaging system according to claim 1, where the small things are made with tungsten of lead.

12. The radiation imaging System according to claim 1 wherein the said openings differ by their depth.

13. The radiation imaging system according to claim 1 wherein the said small things are prismatic with square base.

14. The radiation imaging system according to claim 1 wherein the said small things are shaped like sphere, cylinder or elipsoid.

15. The radiation imaging system according to claim 1 wherein the said small things are shaped like rod standing from the detector to the entrance face of the collimator.

16. The radiation imaging system according to claim 1 comprising a detector of the semiconductor type.
